(19) United States
(12) Patent Application Publication
(10) Pub. No.: US 2001/0051352 A1
(43) Pub. Date: Dec. 13, 2001

(54) MULTILAYER TEST STRIP

(76) Inventors: GARY KRANTZ, LAGUNA BEACH, CA (US); S.T. CHEN, IRVINE, CA (US); ADAM ZIPP, LAGUNA HILLS, CA (US); JOANNE ZENG, SANTA ANA, CA (US)

Correspondence Address: GREGORY O. GARMONG, P.O. BOX 12460, ZAPHYR COVE, NV, 89448

(21) Appl. No.: 08/048,657

(22) Filed: Apr. 15, 1993

(51) Int. Cl. C12Q 1/54

(52) U.S. Cl. 435/14

(57) ABSTRACT

A multilayer test strip includes a first layer having an indicator reagent within a porous substrate, a second layer, overlying the first layer, including a reactive reagent, and a third layer, overlying the second layer, including a spreading agent that promotes the spreading of a liquid upon the third layer. Alternatively, the spreading agent may be mixed into the second layer. An inert backing with an aperture therethrough is positioned over these layers, with the substrate remote from the cover layer.

### BACKGROUND OF THE INVENTION

This invention relates to a test strip for use in testing liquids, and, more particularly, to a multilayer test strip of particular value in optical reflectance testing for the amount of glucose in blood by contacting a sample of the blood to the test strip.

A reagent-containing test strip provides a convenient and easy approach for conducting certain types of chemical analysis. The test strip is normally prepared by impregnating a porous material with a solution containing the reagent, and optionally other ingredients. This step is readily accomplished by dipping the porous material into the reagent solution. The test strip and solution are dried to fix the reagent in the porous matrix. Additional reagents may be added by impregnating the porous material with additional solutions containing the additional reagents and drying the porous material after each further treatment.

To conduct a test using the test strip, a liquid that may contain an active species is contacted to the test strip. The liquid dissolves reagents that were previously introduced into the porous material, so that these reagents can mix with each other and with the active species, if present. The subsequent reaction is selected to cause some measurable change in the test strip. For example, if one of the reagents is a dye, the test strip can be made to change color responsive to the presence of the reactive species.

A test strip may be used in several ways. One common approach is to dip the test strip into a sample of the liquid to be tested, and to thereafter observe the behavior of the test strip. In another approach, a droplet of the liquid is placed onto the test strip. The test strip is observed either with the eye or with an instrument structured and calibrated to make accurate measurements of the test strip.

In one such approach, described in U.S. Pat. No. 4,935,346, a test strip is affixed to an inert backing that has an aperture therethrough to the test strip. A droplet of a liquid to be analyzed, preferably blood in the case of the '346 patent, is placed in the aperture through the backing. The blood penetrates into a first side of the test strip, reacting with reagents in the test strip. The reagents include a dye that colors responsive to the presence of a particular active species in the liquid.

As this test is performed, the test strip and the backing are held in a measurement instrument that directs a light beam against an opposite, second side of the test strip. The light reflected from the second side is measured. Changes in the reflected light quantitatively indicate the presence of the active species in the fluid. The values obtained by this approach are usually not quite as accurate as those obtained by conventional wet chemical analysis. The test strip approach has the important advantage, however, that it can be used by a relatively unskilled person to obtain a close approximation of the amount of the active species present in the fluid sample, with the testing performed in a home or other location away from the chemical laboratory.

This testing approach has been developed into a successful commercial product. However, several shortcomings in the test strips themselves and the methodology of the testing have been observed. These shortcomings can result in inaccuracies in the measured final results. One problem is that the test strips have a relatively short use life. The test strips are normally packed in a hermetically sealed container with a desiccant to keep them dry. The shelf life of the test strips at ambient temperature is about 2 years before the container is first opened, but the use life tends to be only about 4 months after the container is opened. Since the test strips are often purchased by persons who attempt to use them after their use life has expired, it is particularly important to extend the use life as much as possible.

Another problem is that the test results tend to vary with the amount and properties of blood that is placed onto the aperture in the backing. Although the instructions may call for a “drop” of blood to be used, a “drop” is not quantitative. Inexperienced persons may supply droplets of varying sizes. The droplets can vary both because of variations in technique and also because the viscosity of the blood of different persons can vary over a considerable range. The commercially available test strips that are used in the approach of the '346 patent have been observed by the present inventors to produce varying results as a function of the size of the “drop” of blood that is used and the viscosity of the blood.

There is a need for an improved test strip to be used in reflectance analysis procedures. The present invention fulfills this need, and further provides related advantages.

### SUMMARY OF THE INVENTION

The present invention provides a test strip which has greater stability after opening than test strips now used in reflected light blood analytical techniques. The test strips of the invention also exhibit less dependence of the test results on the volume and viscosity of the blood sample than found in conventional test strips. The present test strips can otherwise be substituted for the conventional test strips.

In accordance with one aspect of the invention, a multilayer test strip comprises a first layer having an indicator reagent impregnated into a porous substrate. A second layer having a reactive reagent overlies the first layer. A third layer, comprising a spreading agent that promotes the spreading of a liquid upon the third layer, overlies the second layer. An inert backing overlies the third layer and has an aperture therethrough to the third layer.

In another embodiment, a multilayer test strip comprises a first layer having an indicator reagent impregnated into a porous substrate. A second layer, comprising a reactive reagent and a substance promoting the spreading of a liquid upon the second layer, overlies the first layer. An inert backing overlies the second layer and has an aperture therethrough to the second layer.

The test strip of the invention has two innovations that represent important advances over the prior test strip. First, the indicator reagent and the reactive reagent are placed into two separate layers, so that they cannot chemically interact during storage prior to use. Such chemical interaction is a primary cause of reduced shelf life and degradation of the operability of the test strip during storage.

Second, a spreading layer is provided to improve the spreading of the liquid sample droplet over the upper layer after it is placed in the aperture of the inert backing. The inventors observed that the variability in the coverage of the aperture with different droplet sizes and liquid properties is a primary cause of the variability in the test results using the prior test strips in reflectance testing. In the present approach, the droplet spreads uniformly over the entire surface of the layer exposed through the aperture in the backing, regardless of the droplet size. This spreading of the droplet and the resulting full, uniform coverage of the aperture contributes to a test result that is more nearly independent of the size and flow properties of the sample droplet.

The present approach provides a significant improvement in dry test strips of the type that is particularly useful in reflectance testing for the presence of active chemical species. Other features and advantages of the present invention will be apparent from the following more detailed description of the preferred embodiment, taken in conjunction with the accompanying drawings, which illustrate, by way of example, the principles of the invention.

### BRIEF DESCRIPTION OF THE DRAWINGS

FIG. 1 is an elevational view of a first embodiment of the test strip of the invention;

FIG. 2 is an elevational view of a second embodiment of the test strip of the invention;

FIG. 3 is a perspective view of the test strip of the invention;

FIG. 4 is an elevational view of a prior art test strip; and

FIG. 5 is a process flow diagram for the preparation of the test strip of the invention.

### DETAILED DESCRIPTION OF THE PREFERRED EMBODIMENTS

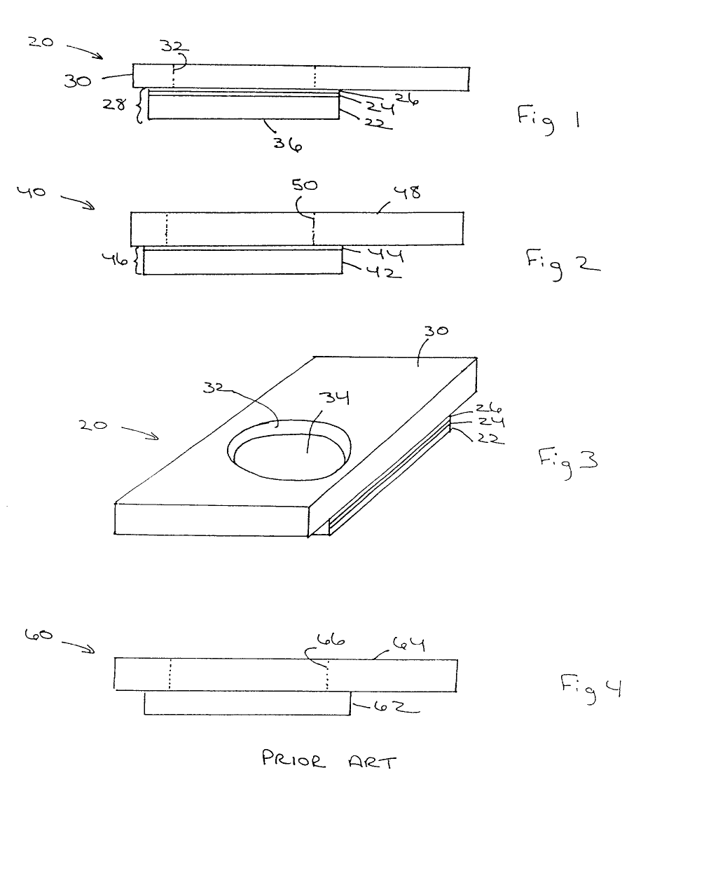

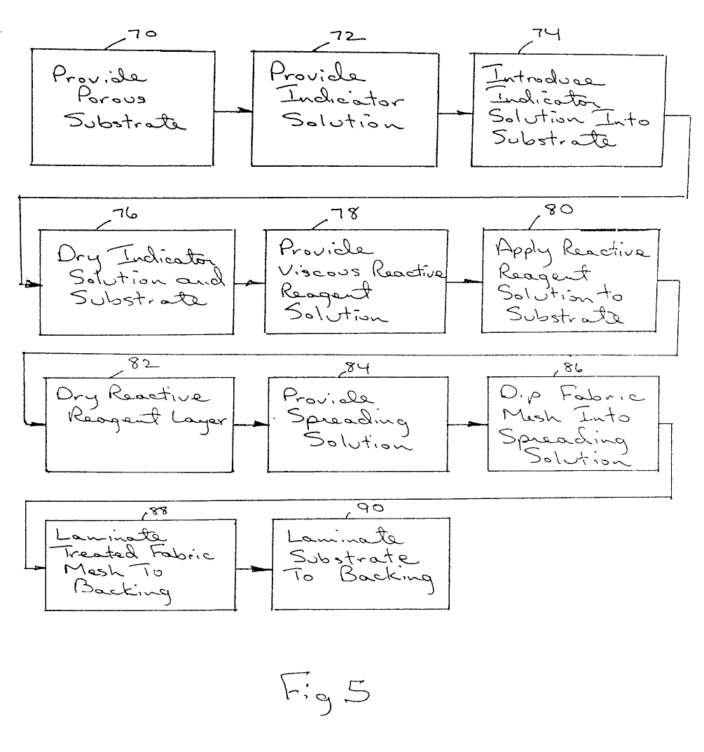

A first preferred embodiment of a test strip 20 according to the invention is illustrated in FIGS. 1 and 3. The test strip 20 includes a porous substrate 22 that is impregnated with a dried indicator formulation, the porous substrate 22 and its indicator formulation acting as a first layer of the structure. Overlying the porous substrate 22 and joined thereto is a second layer 24 that includes a dried reactive reagent formulation. Overlying the second layer 24 and joined thereto is a third layer 26 that includes a dried spreading agent formulation. The three layers 22, 24, and 26 are distinct, physically identifiable layers. There may be some slight intermixing between the layers due to the method of fabrication, to be described later, but generally the layers remain distinct.

The layers 22, 24, and 26 form an active unit 28, which is supported by and joined to a backing 30. The third layer 26 containing the spreading material is adjacent to the backing 30, and the porous substrate 22 containing the indicator formulation is remote from the backing 30. The layers 22, 24, and 26 and the backing 30 must be in this indicated order.

The backing 30 has an aperture 32 therethrough that registers with the layers 22, 24, and 26 of the unit 28. The aperture 32 is indicated with dotted lines in FIG. 1, and is visible in the perspective view of FIG. 3. The third layer 26 is visible through the aperture 32 from an upper side of the test strip 20. As may be seen in FIG. 3, the aperture 32 in combination with the thickness of the backing 30 forms a well 34.

When the test strip 20 is used in its preferred application of glucose testing of blood, it is first placed Into a reflective measurement apparatus of the type disclosed in U.S. Pat. No. 4,935,346, with the aperture 32 facing upwardly. (The disclosure of U.S. Pat. No. 4,935,346 is incorporated herein by reference.) A droplet of blood, whose glucose content Is to be measured, is placed into the well 34 formed by the aperture 32. The spreading formulation of the third layer 26 is dissolved into the liquid, and aids in causing the droplet to spread over the entire surface of the third layer 26 that is exposed through the aperture 32.

The inventors have observed that, in the absence of the spreading formulation, the extent of the spreading of the droplet over the exposed surface of the third layer 26 varies with the size of the droplet that is placed into the well 34, the viscosity of the droplet, and the placement of the droplet. If a large droplet is placed into the well, the entire surface may be covered. If a smaller droplet is placed into the well or the placed droplet partially misses the well, only a portion of the surface of the apertured area may be covered. This variation in coverage leads to a variation in the final measured results of the test, because the optical reflectance instrument has a finite optical spot size on the porous substrate first layer 22 that may encompass both covered and uncovered regions.

In the present approach, by contrast, the spreading formulation aids in spreading small and/or misplaced droplets over the entire surface exposed through the aperture 32. Consequently, the test results are much less dependent upon the volume of the droplet than in the prior approach. This lower volume dependence of the test results is important, in view of the use of the test strip with the reflective measurement apparatus. These products are sold primarily for home diagnostic and measurement use by persons who have little or no scientific or medical training. It is difficult to ensure that these patients will always apply exactly the right amount of liquid in the test, and the present advance is important for these persons.

The presence of the spreading formulation also assists a person who may not deposit the droplet of blood precisely into the well 34. That is, many persons requiring glucose testing are infirm or elderly, and cannot easily draw a droplet of blood from themselves and place it precisely into the well. If the droplet is on the boundary of the well, the spreading layer spreads the droplet across the entire extent of the well.

After the droplet is placed into the well 34, the blood percolates downwardly through the second layer 24, where it dissolves the reagent formulation so that it may intermix with the liquid and the possible reactive substance therein. If the reactive substance is present, it reacts with the reagent formulation to form a reaction product. The reaction product mixes with the liquid of the droplet.

The droplet, with reaction products if any, percolates downwardly further into the porous substrate 22. In the porous substrate 22, the liquid dissolves indicator formulation into the droplet. If the reactive substance was originally present so that reaction product is present in the droplet at this point, the reaction product mixes with the dissolved indicator formulation and reacts therewith.

The reacted indicator formulation is detected by an appropriate technique. In the preferred approach, a light beam is directed against a back side 36 of the porous substrate 22. The light reflected from the back side 36 of the porous substrate 22 is analyzed to determine the presence of the reacted indicator formulation, which in turn is an indication of the presence and amount of the reactive substance in the original droplet. Complete details of the analytical instrument and the procedures are presented in U.S. Pat. No. 4,935,346. Further details of the chemical formulations of the various layers in the test strip 20 are presented subsequently.

FIG. 2 presents a second embodiment of the present invention. Its structure is generally similar to that of the test strip 20 of FIGS. 1 and 3, but differs in the following ways. A test strip 40 is formed of a porous substrate 42 with a second layer 44 overlying the porous substrate 42. The porous substrate 42 and its indicator are identical with the porous substrate 22 and its indicator. The second layer 44, however, contains both the reagent formulation found in the layer 24 of FIG. 1, and the spreading formulation found in the layer 26 of FIG. 1. As with the embodiment of FIG. 1, the porous substrate 42 and the second layer 44 are distinct, separately identifiable layers, which may be slightly intermixed during the manufacturing operation.

The porous substrate 42 and the second layer 44 are formed as an integral unit 46. This unit is attached to a backing 48, with the second layer 44 adjacent to the backing 48 and the porous substrate 42 remote from the backing 48. The backing 48 has an aperture 50 therethrough, comparable with the aperture 32 of FIGS. 1 and 3.

The test strip 40 is used in the same manner as the test strip 20, and as previously described.

In the discussion of the test strips 20 and 40, it has been emphasized that the indicator formulation is placed into one distinct layer, the porous substrate 22 or 42, and the reagent formulation is placed into a separate, distinct layer 24 or 44. This structure is to be contrasted with the structure of a prior test strip 60, shown in FIG. 4. The test strip 60 has a porous substrate 62 joined to a backing 64 with an aperture 66 therethrough. Both an indicator formulation and a reagent formulation are impregnated into the porous substrate 62. The test strip 60 is used in substantially the same manner as the test strips 20 and 40.

Because the indicator formulation and the reagent formulation are mixed together within the porous substrate 62 of the test strip 60, they may partially react and degrade in performance during storage. Such a reaction is most likely in the presence of moisture. The manufacturer normally takes care to package the test strips inside a hermetically sealed container with a desiccant present to reduce moisture. Degradation of performance of the test strips during this sealed period occurs at a moderate rate. After the package is opened, the reaction between the indicator formulation and the reagent formulation is accelerated by the presence of moisture in the air or that otherwise may reach the test strips. Degradation is substantially accelerated after the container is opened.

In the present approach, by contrast, the indicator formulation and the reagent formulation are intentionally separated into two layers. Any degrading interaction occurs, if at all, more slowly than in the prior approach. The test strips 20 and 40 are to be marketed inside sealed containers with a desiccant, like the test strips 60. In the present approach, the multilayer test strips 20 and 40 degrade much more slowly than the single layer test strips 60, both before the packaging is opened and after it is opened. There is a finite shelf life and use life of the test strips 20 and 40, because the reactive formulation and the indicator formulation themselves can degrade without contact with the other, but the shelf life and use life are longer than with the single layer test strips 60.

Mixing the spreading formulation with the reagent formulation in the embodiment of FIG. 2 does not adversely affect shelf life, because these two formulations do not chemically interact. Therefore contact may be made in the layer 44 without loss of shelf life or use life.

The procedure for preparing the test strip 20 of FIGS. 1 and 3 is depicted in FIG. 5. A porous substrate 20 is first provided, numeral 70. The porous substrate 20 may be that described in the '346 patent at col. 4-6. In the preferred form of the present invention, the porous substrate is a piece of nylon membrane having a thickness of about 0.005-0.0075 inches and lateral dimensions of about 0.3 by 0.6 inches. This porous substrate material is available commercially from Pall Corp. as Biodyne A. This material is made from nylon 66 and has a porosity of about 5 micrometers.

A solution containing the indicator is provided, numeral 72. The preferred indicator is a dye whose appearance changes after a chemical reaction. A preferred indicator solution has a composition (for 100 milliliters of solution) of 1 gram DMAB (3-dimethylaminobenzoic acid), 30 milliliters of DMSO (dimethylsulfoxide), 0.33 grams Na₂HPO₄, 0.63 grams NaH₂PO₄, and 70 milliliters deionized water. The invention is not limited to this preferred indicator.

The indicator solution is introduced into the porous substrate 20, numeral 74. The above-described indicator solution is formulated to have a sufficiently low viscosity that it may be readily introduced by dipping the porous matrix into the solution for 30 seconds at ambient temperature, and permitting the excess solution to drain away.

The porous substrate 20 and its loading of indicator solution are dried, numeral 76, to remove the solvent from the porous substrate. Drying is preferably accomplished in air at 56C for 20 minutes. The indicator formulation is thereby dried and adhered to the fibers of the porous substrate. A solution containing the reactive reagent formulation is provided, numeral 78. The preferred reactive reagent for glucose determination is a mixture of glucose oxidase and peroxidase. A preferred composition for the reactive reagent solution is 18000 I.U. glucose oxidase, 8040 I.U. peroxidase, 0.13 grams MBTH (3-methyl-2-benzothiazolinone hydrazone hydrochloride), 0.02 grams EDTA (ethylenediaminetetraacetic acid), 0.08 grams gelatin, 0.21 grams Na₂HPO₄, 0.46 grams NaH₂PO₄, 0.3 grams PVP (polyvinylpyrrolidone), 0.067 grams xanthan gum, 0.18 grams deoxycholic acid sodium salt, 0.1 grams DOBS (dodecylbenzene sulfonate sodium salt), and 26.4 milliliters distilled water.

The reactive reagent solution is applied as the separate layer 24 onto the porous substrate 20, numeral 80. The above-described reagent solution is prepared to have a sufficiently high viscosity that It may be readily coated as a layer onto the surface of the porous substrate 20, without penetrating into the porous substrate 20. The resulting layer 24 has a thickness of about 0.001 inch, when applied. Because the reactive reagent solution does have a liquid form, there is some interpenetration into the porous substrate 20, but that Interpenetration is quite small. Such a slight degree of interpenetration is desirable, as it aids in adhering the layer 24 to the porous substrate 22 upon subsequent drying.

The reactive reagent layer is dried, numeral 82, to remove the solvent from the reactive reagent formulation. Drying is preferably accomplished in an oven at 60 C. The reactive reagent formulation is thereby dried is the layer 24 which is adhered to the surface of the porous substrate 22.

A solution containing the spreading formulation is provided, numeral 84. The preferred spreading solution is a 0.2 percent solution of polyoxyethylene ether.

A piece of polyester fabric mesh is dipped into the spreading solution and dried, numeral 86. Drying is preferably accomplished in an oven at 60 C.

The treated piece of fabric mesh is laminated to the backing 30, numeral 88. The backing is preferably a piece of polyvinylchloride having a thickness of about 0.005-0.010 inches. An aperture having a diameter of about 5 millimeters is preformed through the backing. The treated piece of fabric mesh becomes the spreading layer or third layer 26 of the final test strip.

To complete the fabrication of the test strip, the laminate of backing 30 and third layer 26 is laminated to the previously prepared substrate 22 and second layer 24, numeral 90. In this laminate, the second layer 24 is adjacent to the third layer 26. During the processing procedures, there may be some slight interpenetration of the layers 24 and 26, but that interpenetration is quite small. Such a slight degree of interpenetration can be desirable, as it aids in adhering the layer 26 to the layer 24.

The pieces are usually prepared as large sheets, and diced into the appropriately sized individual test strips. The resulting structure is as shown in FIGS. 1 and 3.

The alternative embodiment of FIG. 2 may be prepared in a manner similar to that of FIG. 5, except that step 84 is combined into step 78, with the reactive reagent and the spreading material in a single solution, and steps 86 and 88 are omitted. A preferred composition for such a combined spreading solution is 0.1 grams EDTA, 0.5 grams gelatin, 22 milliliters of 1 molar buffer (pH 6.0, Na₂HPO₄ and NaH₂PO₄), 7.5 milliliters of 20 percent polyvinylpyrrolidone, 22 grams of 1.5 percent xanthan gum, 5 milliliters of 10 percent detergent such as dodecylbenzene sulfonate sodium salt, 5 grams of diatomaceous earth, 0.4 grams MBTH, 108,000 I.U. glucose oxidase, and 52,000 I.U. of peroxidase. This solution is applied overlying the porous substrate 22, to which the indicator solution (having the composition already discussed) has already been Introduced and dried. The single solution is dried. The resulting structure has two layers fixed to the backing, as shown in FIG. 2.

Samples of the test strips have been prepared and tested to determine the glucose oxidase content of blood. Test strips prepared according to the present invention were tested both by themselves and comparatively with the prior test strips as shown in FIG. 4. All measurements were performed using the “One Touch” optical measurement instrument manufactured by Lifescan, Inc., Milpitas, Calif., generally in accordance with U.S. Pat. No. 4,935,346, and its recommended procedure. The following results illustrate aspects of the invention, but should not be taken as limiting of the invention in any respect.

### EXAMPLE 1

Use Life

Multilayer test strips having the structure of FIG. 2 and prepared as described above were tested to determine changes in performance due to exposure to degrading environments. Ten test strips were tested in each group, and the reported results are averages of the results of the eight test strips in each group. Comparative testing was performed with groups of single layer commercial “One Touch” test strips, having a structure generally like that of FIG. 4. The testing was as follows. A first group A of each type of test strip was tested immediately. A second group B was exposed to ambient temperature air for three days. A third group C was heated to 60 C for three days in an accelerated degradation test. The blood glucose content of blood drawn from a single sample was measured using each of the test strips. The results are as follows, with blood glucose expressed in milligrams per deciliter.

| | Measured Blood Glucose | | |
|---|---|---|---|
| Test Strip | Group A | Group B | Group C |
| Present Multilayer | 231 | 230 | 233 |
| Prior Single Layer | 231 | 207 | 198 |

The multilayer and single layer test strips gave the same reading prior to atmospheric exposure, Group A. After atmospheric exposure for three days, Group B, the multilayer test strip gave a reading that stayed substantially constant, while the single layer test strip had a significant change of about 10 percent in its reading. After the accelerated testing of Group C, the multilayer test strip gave essentially the same reading, but the single layer test strip suffered an even larger change in reading. These results are important because a doctor cannot know the exact state of the test strip used by the patient in home testing, and changes due to environmental exposure can affect the doctor's interpretation of the results.

### EXAMPLE 2

Hematocrit Dependence

Hematocrit is a measure of the blood viscosity of a person. The greater the hematocrit, the more viscous is the blood and the greater difficulty in causing the blood to spread across a surface. To test the significance of hematocrit in relation to the type of test strip, blood was prepared with an artificially introduced variation in hematocrit. Blood was diluted with plasma from the sample to produce artificial “blood” samples with hemaLocrits of 65%, 50%, 37%, and 27%. Two different glucose ranges were tested, a high range with a glucose of over 100 and a low range with a glucose of less than 100. The various blood hematocrit values (HCT) were tested for glucose content using the two types of test strips. Blood serum was prepared from the blood and tested for blood glucose content using the test strips to establish a measured 0 percent HCT value. The following table reports the results for the various blood hematocrit values that were tested.

| Test Strip | Measured Blood Glucose | | | | |
|---|---|---|---|---|---|
| HCT%: | 0 | 27 | 37 | 50 | 60 |
| Present Multilayer | | | | | |
| High Glucose | 329 | 314 | 309 | 278 | 245 |
| Low Glucose | 106 | 96 | 85 | 89 | 69 |
| Prior Single Layer | | | | | |
| High Glucose | 192 | 308 | 262 | 198 | 115 |
| Low Glucose | 78 | 89 | 82 | 62 | 50 |

Both test strips exhibit some dependence of the blood glucose content on blood HCT value. However, the multilayer test strip has a much lower dependence of its indicated blood glucose content on the hematocrit value than does the single layer test strip. The lesser dependence is particularly noticeable for the high blood glucose samples. The practical consequence of this result is that the present multilayer test strip gives results that are less dependent upon HCT value than does the prior single layer test strip.

Although particular embodiments of the invention have been described in detail for purposes of illustration, various modifications may be made without departing from the spirit and scope of the invention. Accordingly, the invention is not to be limited except as by the appended claims.

What is claimed is:

1. A multilayer test strip, comprising:
a first layer comprising an indicator reagent impregnated into a porous substrate;
a second layer, overlying the first layer, comprising a reactive reagent;
a third layer, overlying the second layer, comprising a spreading agent that promotes the spreading of a liquid upon the third layer; and
an inert backing overlying the third layer, the backing having an aperture therethrough to the third layer.

2. The test strip of claim 1, wherein the porous substrate is a nylon membrane.

3. The test strip of claim 1, wherein the indicator is DMAB.

4. The test strip of claim 1, wherein the reactive reagent is selected from the group consisting of glucose oxidase and peroxidase.

5. The test strip of claim 1, wherein the spreading agent is selected from the group consisting of cellulose acetate and diatomaceous earth.

6. A multilayer test strip, comprising:
a first layer comprising an indicator reagent impregnated into a porous substrate;
a second layer, overlying the first layer, comprising a reactive reagent and a substance promoting the spreading of a liquid upon the second layer; and
an inert backing overlying the second layer, the backing having an aperture therethrough to the second layer.

7. The test strip of claim 6, wherein the porous substrate is a nylon membrane.

8. The test strip of claim 6, wherein the indicator is DMAB.

9. The test strip of claim 6, wherein the reactive reagent is selected from the group consisting of glucose oxidase and peroxidase.

10. The test strip of claim 6, wherein the spreading agent is selected from the group consisting of cellulose acetate and diatomaceous earth.

11. A multilayer test strip, comprising:
a first layer comprising an indicator reagent impregnated into a porous substrate, wherein the indicator reagent is DMAB;
a second layer, overlying the first layer, comprising a reactive reagent that reacts with glucose in human blood, the reactive reagent being selected from the group consisting of glucose oxidase and peroxidase, and
a third layer, overlying the second layer, comprising a spreading agent that promotes the spreading of a droplet of blood upon the third layer; and
an inert backing overlying the third layer, the backing having an aperture therethrough to the third layer.

12. A multilayer test strip, comprising:
a first layer comprising an indicator reagent impregnated into a porous substrate, wherein the indicator reagent is DMAB;
a second layer, overlying the first layer, comprising
a reactive reagent that reacts with glucose in human blood, the reactive reagent being selected from the group consisting of glucose oxidase and peroxidase, and
a spreading agent that pronotes the spreading of a droplet of blood upon the second layer; and
an inert backing overlying the second layer, the backing having an aperture therethrough to the second layer.

13. A test strip, comprising:
a reaction unit having a spreadability enhancing material at a top surface thereof; and
a backing having an aperture therein, to which the reaction unit is affixed with the top surface contacting the backing.

14. A method for preparing a test strip, comprising the steps of:
providing a porous substrate;
providing an indicator solution containing the indicator DMAB, the indicator solution having a sufficiently low viscosity that it is penetrable into the porous substrate;
introducing the indicator solution into the porous substrate;
drying the indicator solution within the porous substrate;
providing a reactive reagent solution containing a reagent selected from the group consisting of glucose oxidase and peroxidase, the solution having a sufficiently high viscosity that it is not penetrable into the porous substrate;
applying the reactive reagent solution as a reactive reagent layer to a surface of the porous substrate;
drying the reactive reagent within the reactive reagent layer; and
attaching the porous substrate to a backing having an aperture therethrough, with the applied layer adjacent to the backing and the porous substrate remote from the backing.

15. The method of claim 14, wherein the step of providing a reactive reagent solution includes the step of
mixing a spreading solution into the reagent solution.

16. The method of claim 14, including the additional steps, after the step of drying the reactive reagent and before the step of attaching the porous substrate, of
providing a spreading solution containing a spreading agent; and
applying the spreading solution as a spreading layer to the reactive reagent layer.
